(19) United States
(12) Patent Application Publication
(10) Pub. No.: US 2021/0322474 A1
(43) Pub. Date: Oct. 21, 2021

(54) MODULATION OF APOPTOSIS SUSCEPTIBLE CELLS

(71) Applicant: CELLECT BIOTHERAPEUTICS LTD., Kfar Saba (IL)

(72) Inventors: Shai YARKONI, Kfar Saba (IL); Hilit LEVI-BARZANI, Kfar Saba (IL)

(73) Assignee: CELLECT BIOTHERAPEUTICS LTD., Kfar Saba (IL)

(21) Appl. No.: 17/270,001

(22) PCT Filed: Aug. 22, 2019
(86) PCT No.: PCT/IL2019/050945

§ 371 Date: Feb. 20, 2021

Related U.S. Application Data
(60) Provisional application No. 62/720,988, filed on Aug. 22, 2018.

(51) Int. Cl. A61K 35/17; C12N 5/0781; C12N 5/0783; C12N 5/00; C07K 14/705

(52) U.S. Cl. CPC A61K 35/17; C12N 5/0635; C12N 2501/48; C12N 5/0087; C07K 14/70503; C12N 5/0636

(57) ABSTRACT

Provided are methods for producing a population of cells enriched with non-activated/non-mature cells, in particular non-activated/non-mature T and/or B cells, optionally genetically modified T and/or B cells. The method includes contacting a heterogeneous population of mammalian cells with an apoptosis inducing ligand, wherein said contacting induces apoptosis of active/mature cells while non active/mature cells remain resistant to the apoptotic signal. Further provided are therapeutic uses of the enriched cell populations.

### TECHNOLOGICAL FIELD

The present invention is in the field of cell therapy.

### BACKGROUND ART

References considered to be relevant as background to the presently disclosed subject matter are listed below:
1. Watkins et al, “Tracking the T-cell repertoire after adoptive therapy”. Clinical & Translational Immunology 6, e140 (2017).
2. Turtle et al, “CD19 CAR-T cells of defined CD4+:CD8+ composition in adult B cell ALL patients”. J. Clin. Invest. 126(6):2123-38 (2016).
3. Lamb et al, “Ex vivo T-cell depletion in allogeneic hematopoietic stem cell transplant: past, present and future”. Bone Marrow Transplantation 52, 1241-1248 (2017).
4. Baecher-Allan et al, “Multiple Sclerosis: Mechanisms and Immunotherapy”, Neuron, 97: 742-768, (2018).
5. Mazar J, et al. Cytotoxicity mediated by the Fas ligand (FasL)-activated apoptotic pathway in stem cells. J. Biol. Chem. 2009; 284:22022-22028.
6. Knight J C, Scharf E L, Mao-Draayer Y. Fas activation increases neural progenitor cell survival. J. Neurosci. Res. 2010 March; 88(4):746-57.
7. Locke et al, “Phase 1 Results of ZUMA-1: A Multicenter Study of KTE-C19 Anti-CD19 CAR T Cell Therapy in Refractory Aggressive Lymphoma”. Molecular Therapy 25; 1 (2017).
8. Sommermeyer et al, Chimeric antigen receptor-modified T cells derived from defined CD8+ and CD4+ subsets confer superior antitumor reactivity in vivo. Leukemia. 30, 492-500 (2016).
9. Bonifant et al, “Toxicity and management in CAR T-cell therapy”. Molecular Therapy Oncolytics 20; 3:16011 (2016).
10. Kim et al, “Human CD34 hematopoietic stem/progenitor cells express high levels of FLIP and are resistant to Fas-mediated apoptosis”. Stem Cells; 20:174-182 (2002).
11. Sprent and Tough, “T Cell Death and Memory”. Science, 293(5528):245-8, (2001).
12. Strasser et al., “The Many Roles of FAS Receptor Signaling in the Immune System”. Immunity, 30(2):180-92, (2009)
13. Zhang et al, “Host-reactive CD8⁺ memory stem cells in graft-versus-host disease”. Nat Med. (2005).
14. Roberto et al, “Role of naïve-derived T memory stem cells in T-cell reconstitution following allogeneic transplantation”. Blood. 125:2855-2864 (2015).
15. Nashi et al, “The Role Of B Cells in Lupus Pathogenesis”. Int J Biochem Cell Biol. 42(4): 543-550, (2010).
16. Baker et al. “Memory B Cells are Major Targets for Effective Immunotherapy in Relapsing Multiple Sclerosis”. EBioMedicine, 16: 41-50, (2017).
17. Hackett et al Mol. Ther. (2010) 18(4): 674-683.
18. MacDonald K P et al, “Biology of graft-versus-host responses: recent insights”. Biol Blood Marrow Transplant. 19(1): S10-S14, (2013).
19. Graham et al. “Allogeneic CAR-T Cells: More than Ease of Access?” Cells (2018) October; 7(10): 155.
20. Xu Y et al. “Closely related T-memory stem cells correlate with in vivo expansion of CAR.CS19-T cells and are presented by IL-7 and IL-15”. Blood. (2014); 3750-3760.

Acknowledgement of the above references herein is not to be inferred as meaning that these are in any way relevant to the patentability of the presently disclosed subject matter.

### BACKGROUND

Multiple cell therapy products, autologous or allogeneic, particularly in cancer therapy, are based on a heterogenic mixture of cells as a raw material (Watkins et al, 2017; Turtle et al, 2016). The isolation and enrichment techniques currently utilized to reduce toxicity of the transplanted cell populations, may result in a more defined, final product, which is lacking some of the desired biological activities due to the non-selective nature of the depletion techniques (Lamb et al, 2017). These isolation techniques utilize mechanical or phenotypic characteristics, often too rough, and not sensitive enough to differentiate between desired and undesired cells within a cell population. A new method to standardize the starting material used for manufacturing of cell-based products is required, to get a final product which is well characterized and reproducible with a defined biological activity. Preferably this method will be used ex vivo, prior to patient treatment with the cell product, to reduce side effects and improve outcome.

Another major goal in cell therapies for selective depletion, for example in the field of autoimmune diseases therapy, is developing effective therapeutic strategies to reduce the activation potential and the pro-inflammatory reaction (Baecher-Allan et al, 2018).

An additional challenge in cell therapy is in the field of regenerative medicine, when the starting material is a heterogeneous population, where the non-effective cells are diminishing the potency of the cell therapy product (Mazar J, 2009: Knight, 2010).

An example of the difficulty in using a heterogenic population in cell therapy is the case of chimeric antigen receptor genetically engineered T (CAR-T) cells. Adoptive T cell therapy (ACT) utilizing CAR-T cells that has been investigated for various anti-tumor treatments may provide an effective way to treat several cancers, since CAR-T cells can be genetically engineered to specifically recognize antigenically-distinct tumor populations (see for example Locke et al, 2017). These T cell-based therapies have been shown in clinical trials to be remarkably promising for highly refractory B-cell malignancies. However, since in most reported trials, patients have received T-cell products comprising random compositions of T cell subsets, each patient received a different therapeutic agent, which may have influenced the efficacy of the T-cell therapy, and complicated comparison of outcomes between different patients and across trials. Recent studies by the group of Riddle & Maloney, in ALL patients, suggest that the CAR-T-cell products generated from defined T-cell subsets can provide uniform potency compared with products derived from unselected T cells, which vary in phenotypic composition. (Turtle et al, 2016; and Sommermeyer et al, 2016).

The CAR-T cell immunotherapy presents major challenge in toxicity management. The two most commonly observed toxicities with CAR-T cell therapies are the CAR-T cell related encephalopathy syndrome (CRES) and the cytokine release syndrome (CRS), which ranges from mild to life threatening, a constellation of inflammatory symptoms resulting from elevated cytokines usually within the first week and peaks within 1-2 weeks of cell administration, and associated with T cell activation and proliferation (Bonifant et al 2016). The risk of toxicity is limiting wide deployment of the CAR-T cell treatment. The current medical strategy for reducing the toxicities related to the CAR-T cell therapy includes post treatment anti-inflammatory modalities. For example, anti-IL6 receptor or an IL6 receptor antagonist, and corticosteroids, both modalities suppress inflammatory responses and are, therefore, effective in the management of CRS and CRES that are associated with the cellular therapies. The drawback however is that these treatments are down regulating the immune response, and their potential to block T cell activation and abrogate clinical benefit is a concern. The challenge in toxicity management is controlling symptoms without compromising efficacy (Bonifant et al, 2016).

An additional challenge is the transduction efficiency. Transduction efficiency is affected by the T cells quality. Activation of the T cells is a pre-requisite for efficient transduction as primary human T cells are non-dividing quiescent cells in vitro. In addition, the quality of T cells of patients which have undergone chemotherapy is compromised. T cell dysfunction is common and frequently cannot be fully reversed during the manufacturing process (Graham et al 2018).

Another challenge concerns post treatment immune down regulation. It is possible that CAR modified T cells will be rendered ineffective upon entering the suppressive tumour microenvironment. This is especially important in the attempts to develop CAR-T cells therapy for solid tumours. Apoptotic signalling within the tumour milieu is down regulating all immune effector cells.

Studies suggest that mature effector cells such as effector memory T cells (EM) are less efficient CAR-T cells in in vivo T cell expansion, survival, persistence and antitumor activity than CAR-T cells manufactured from early differentiated, less mature T cells, mostly naïve and central memory (CM) (Sommermeyer D, 2016, and Xu Y, 2014).

WO2013/132477 discloses devices and methods for selecting apoptosis-signaling resistant cells comprising exposing immune cell populations to an apoptosis-inducing ligand.

### GENERAL DESCRIPTION

In a first of its aspects, the present invention provides a method for producing a population of cells enriched with non-activated/non-mature cells, comprising:
a. obtaining a biological sample comprising a heterogeneous population of mammalian cells;
b. contacting the obtained heterogeneous population of mammalian cells with an apoptosis inducing ligand in a container, wherein said contacting induces apoptosis of active/mature cells while non active/mature cells remain resistant to the apoptotic signal, thereby isolating a population of cells enriched for non-active/non-mature cells.

In one embodiment, said mammalian cells are human cells.

In another embodiment, said mammalian cells are selected from the group consisting of immune cells and multipotential stromal/mesenchymal stem cells.

In one embodiment, said non active/non-mature cells are immune cells.

In one embodiment, said non active/non-mature cells are naïve-immune cells.

In one embodiment, said container comprises a physiological solution and/or a growth medium, and/or autologous or non-autologous human plasma.

In a specific embodiment, the present invention provides a method for producing a population of cells enriched with naïve-immune cells, comprising:
a. obtaining a biological sample comprising a heterogeneous population of mammalian immune cells; and
b. contacting the obtained heterogeneous population of mammalian immune cells with an apoptosis inducing ligand in a container, wherein said contacting induces apoptosis of mature cells while naïve cells remain resistant to the apoptotic signal, thereby isolating a population of cells enriched for naïve cells.

In one embodiment, said naïve-immune cells are naïve-T cells or naïve-B cells.

In another embodiment, said biological sample is selected from the group consisting of mobilized peripheral blood cells, peripheral blood mononuclear cells (PBMC), enriched CD3⁺ T cells, enriched CD4⁺ or CD8⁺ T cell, enriched B cells, cord blood cells and bone marrow cells.

In one embodiment, said immune cells are autologous to the patient or allogenic to the patient.

In one embodiment, said container comprises a physiological solution and/or a growth medium, and/or autologous or non-autologous human plasma.

In one embodiment, the apoptosis inducing ligand is immobilized on an inner surface of the container or on beads or films comprised in the container.

In one embodiment, the apoptosis inducing ligand is selected from the group consisting of TNF-α, Fas ligand (FasL), TRAIL and TWEAK.

In one embodiment, said contacting step with an apoptosis inducing ligand is performed for between about 1 hour to about 48 hours.

In one embodiment, said contacting step is performed for about 2 hours.

In one embodiment, said apoptosis inducing ligand is FasL and wherein said FasL is administered in a concentration of between about 1 to about 800 ng/ml.

In one embodiment, FasL is administered at a concentration of about 100 ng/ml.

In one embodiment. FasL is administered at a concentration of about 10 ng/ml.

In one embodiment, said mature cells are mature T cells selected from the group consisting of T_{H}1/T_{C}1, T_{H}17, T_{SCM}, T_{CM}, T_{EM}, and T_{eff} cell populations.

In another aspect, the present invention provides, a population of cells enriched for naïve-T cells prepared by the method of any one of the preceding claims.

In one embodiment, said cells enriched for naïve-T cells are characterized as CCR7⁺CD45RA⁺CD95-LFA1^{low}.

In another aspect, the invention provides the population of cells enriched for naïve-T cells of the invention for use in the treatment of cancer and autoimmune diseases.

In another aspect, the invention provides the population of cells enriched for T cells that maintain their activation potential as a pre-requisite for genetic modification, for use in the treatment of cancer and autoimmune diseases.

In another aspect, the invention provides a method of treating autoimmune diseases in a patient comprising administering to said patient a population of cells enriched for naïve-T cells prepared by the methods of the invention.

In one embodiment, said mature cells are mature B cell populations selected from the group consisting of memory and plasmablast B cell populations.

In another aspect, the present invention provides a population of cells enriched for naïve-B cells prepared by the methods of the invention.

In one embodiment, said naïve-B cells are characterized as CD27⁺CD38⁺.

In another aspect, the present invention provides the population of cells enriched for naïve-B cells of the invention for use in the treatment of cancer, autoimmune diseases, or inflammatory diseases.

In another aspect, the present invention provides a method of treating autoimmune diseases in a patient comprising administering to said patient a population of cells enriched for naïve-B cells prepared by the methods of the invention described herein.

In another aspect, the present invention provides a method of treating autoimmune diseases comprising:
a. contacting a heterogeneous population of mammalian immune cells comprising T and B cells with an apoptosis inducing ligand, wherein said contacting reduces the activation level of said T and B cells: and
b. administering said population of cells obtained in step (a) into a patient in need thereof.

In another aspect, the present invention provides a method of treating cancer in a patient comprising administering the population of cells enriched for non-mature T cells of the invention, wherein said cells preserve their anti-cancer activity.

In another aspect, the present invention provides a method for producing CAR-T cells, comprising:
a. isolating mononuclear cells from a biological sample;
b. activating the cells by contacting said cells with at least one T cell activating agent; and
c. Transducing said cells with a CAR construct;
   wherein said method further comprises contacting said cells with an apoptosis inducing ligand before the activating step (b) and/or after the transducing step (c), thereby obtaining CAR-T cells.

In one embodiment, said mammalian cells are human cells.

In one embodiment, said biological sample is selected from the group consisting of peripheral blood mononuclear cells (PBMC), enriched CD3⁺ T cells, enriched CD4+ T cells, enriched CD8⁺ T cells and any combination thereof.

In one embodiment, said cells are PBMC.

In one embodiment, said T cell activating agents are anti-CD3 and anti CD28 antibodies.

In one embodiment, the apoptosis inducing ligand is selected from the group consisting of FasL, TNF-α, TRAIL and TWEAK.

In one embodiment, said contacting step with an apoptosis inducing ligand is performed for between about 1 hour to about 48 hours.

In one embodiment, said contacting step is performed for about 2 hours.

In one embodiment, said apoptosis inducing ligand is FasL and said FasL is administered in a concentration of between about 1 to about 800 ng/ml.

In some embodiments, FasL is administered at a concentration of about 10 ng/ml, 50 ng/ml or 100 ng/ml.

### BRIEF DESCRIPTION OF THE DRAWINGS

In order to better understand the subject matter that is disclosed herein and to exemplify how it may be carried out in practice, embodiments will now be described, by way of non-limiting example only, with reference to the accompanying drawings, in which:

FIG. 1, is a set of graphs (1A-1G) showing expression levels of CD95 (FasR), on the surface of T cell subtypes. T-cells (CD3⁺) derived from G-CSF Mobilized Peripheral Blood Cells (MPBC) graft were characterized by flow cytometry. (A) CD3⁺ cells; (B) CD4⁺ cells; (C) Various CD4⁺ subtypes: Naïve, T stem cell memory (T_{SCM}), central memory (CM), effector memory (EM), effector (eff); (D) mature T cells of the subtypes TH1, TH17; (E) CD8⁺ cells; (F) Various CD8⁺ subtypes: Naïve, T_{SCM}, CM, EM, eff; (G) mature T cells of the subtype T_{C}1.

FIG. 2, is a set of graphs (2A-2G) showing in (A-G) immuno-phenotype based profiling of T cell subtypes population percentages in Fas-L treated MPBC, compared to MPBC control. 7AAD⁺ (necrotic/late apoptotic) cells were excluded from the analysis. (A) CD4⁺ T helper (T_{H}); (B) Various CD4⁺ subtypes: Naïve. T_{SCM}, CM, EM and eff; mature pro-inflammatory T cells (C) TH1 (D) T_{H}17: (E) CD8⁺ T cytotoxic (TC); (F) Various CD8⁺ subtypes; (G) mature pro-inflammatory T cells: TC1; (H-N) are graphs showing the early apoptosis level of Fas-L treated cells evaluated by flow cytometry using Annexin V⁺7AAD⁻ staining and compared to control MPBCs. Results are presented as Mean+SD of representative experiment out of 3 independent experiments with triplicates. (H) CD4⁺ cells; (I) Various CD4⁺ subtypes: Naïve, T_{SCM}, CM, EM, eff; (J) mature T cells of the subtype TH1. (K) mature T cells of the subtype TH17; (L) CD8⁺ cells; (M) Various CD8⁺ subtypes: Naïve, T_{SCM}. CM. EM, eff; (N) mature T cells of the subtype TC1;

(O) and (P) are graphs showing expression of CD25 receptor (activation marker) as measured in FasL treated T helper (CD4⁺ CD25⁺) cells (O), and T cytotoxic (CD8⁺CD25⁺) cells (P), compared to MPBCs control using flow cytometry. (Q) Percentage of regulatory T cells (Tregs) (CD127-) out of CD4⁺CD25⁺ activated helper T cells following FasL treatment compared to MPBCs control. Mean+SEM, n=11 independent grafts. Statistical analysis was made using non-parametric, paired Student's T test *P<0.05, **P<0.01, ***P<0.001 ****P<0.0001.

FIG. 3, is a set of graphs showing reduced activation of Fas-L treated T lymphocytes in response to in-vitro activation. T-lymphocytes isolated from Fas ligand treated mobilized peripheral blood cells and control cells were incubated at 0.75×10⁶ cells/ml and stimulated using CD3/CD28 activation beads, at 1:10 bead:cell ratio, for 24 or 48 hrs. CD25^{high} receptor expression was measured in Fas-L treated T helper (CD4⁺CD25^{high}) (A) and T cytotoxic (CD8⁺CD25^{high}) cells (B), and compared to MPBCs control using flow cytometry. (C) IFNγ secretion by the Fas-L treated and control cells was measured using ELISA. Results are representative of two independent studies. Data presented as Mean+SD, n=3 replicates. Statistical analysis was performed using unpaired, parametric t-test: (D) Fas-L treated human MPBCs (5×10⁶) were transplanted to 3 cohorts of Sub-lethally irradiated (2Gy) NSG mice and compared to MPBC controls (n=8-10/group) or vehicle (transplantation buffer, n=2/group). At three termination time points (3, 7 and 14 days post transplantation) the absolute hCD3⁺ T cells number in the spleen (calculated by multiplying hCD3⁺ T cells percentage, as detected by flow cytometry, with the absolute number of cells harvested from each tissue). (E) Kaplan Maier survival curve (graft versus host disease (GvHD) survival curve). (F) Serum levels of IFN-γ at day 14 post transplantation. Results are representative of two independent studies (n=8-10 recipients/group). Data presented as Mean+SEM. statistical analysis performed using Mann Whitney test: *P≤0.05, **P≤0.01, ***P≤50.001, ****P≤50.001.

FIG. 4, is a set of graphs showing that the Fas-L treatment, followed by reduction of mature cells populations, does not affect graft versus leukemia activity both in-vitro and in-vivo. Cytotoxic activity assay of MPBC-control or Fas-L treated MPBCs towards (A) U937 (B) MV4-11 leukemic cell lines. 2×10⁴ CFSE labeled-leukemic cells/well were cultured in 96-well plate, expanded T-cells (12 day culture with anti CD3 and recombinant IL2) were added in elevated ratio of Leukemia:T-cells. Viable CFSE-leukemic cells were assessed after 24 hours of co-culture by FACS. Data presented as Mean+SD, n=3 replicates. (C) NOD-scid IL2Rgamma-null (NSG) mice were γ-irradiated (200cGy) on day (−1), 10×10\hat{ }6 MV4-11 leukemic cells were administered on day 0 by intravenous (IV) bolus injection. 4-6 hrs later, 3×10\hat{ }6 MPBCs or FasL treated MPBCs were administered by IV bolus injection. Animals were scored twice a week. Assessment of human hematopoietic cell engraftment (CD45⁺CD123⁻) and the leukemic burden (CD45⁺CD123⁺) were assessed 3 weeks post transplantation in the (D) spleen, (E) bone marrow and (F) blood by flow cytometry. Data presented as Mean+SEM, n=7 female NSG mice per group. Representative results of one out of two independent experiments. *P<0.05, **P<0.01 versus Vehicle treated group and #P<0.05, ##P<0.01 versus MPBCs control group (Mann Whitney test).

FIG. 5, is a set of graphs showing the effect of Fas-L treatment on Antigen Presenting Cells (APCs)—B cells and myeloid cells both in-vitro and in-vivo. (A) The level of FasR (CD95⁺) expression of MPBC control cells was measured using flow cytometry, (n=3). (B) Apoptotic cell percentage (Annexin V⁺ stained cells, n=8) and percentage of HLA-DR^{hi} expressing cells (triplicate, n=1) was detected in FasL treated MPBCs compared to control MPBCs: (C) percentage of HLA-DR⁺ of CD19⁺ cells and D) percentage of HLA-DR⁺ of CD33⁺ cells. (E-L) NSG mice were transplanted with FasL treated MPBCs or MPBC controls (5×10⁶ total nucleated cells (TNCs)/mouse) (n=8-10/group). At each indicated termination time point (days 3/7/14) the spleen and bone marrow were collected and the absolute number of B cells and myeloid cells was detected in the spleen (E) and (F); and bone marrow (I) and (J). Percentage of HLA-DR^{hi} expressing B and myeloid cells in the spleen (G) and (H) and bone marrow (K) and (L) of FasL treated MPBC transplanted mice, compared to MPBC-control transplanted mice. Data presented as Mean+SEM. Statistical analysis was performed using student's t-test: (B, paired, non-parametric); (C. D unpaired, parametric) (E-L, unpaired, non-parametric); *P<0.05, **P<0.01, ***P<0.001, ****P<0.0001.

FIG. 6, is a set of graphs showing the distribution of B cell subtypes in G-CSF mobilized PBCs graft, their expression of FasR and response to apoptosis induction by Fas-L. (A) The level of FasR (CD95⁺) expression on B cell subtypes according to their maturation stage (Transitional/Naïve/Memory and Plasmablast) in G-CSF mobilized peripheral blood samples using flow cytometry was measured. (B) The early apoptotic level of the B cell subtypes in Fas-L treated MPBC was evaluated by flow cytometry using Annexin V⁺7AAD⁻ staining and compared to control MPBCs. (C) Immunophenotype profiling of B cells subtypes of both Fas-L treated and control MPBC. 7AAD⁺ (necrotic/late apoptotic) cells were excluded from the analysis. Data presented as Mean+SD, of triplicates. statistical analysis was performed using unpaired, parametric t-test: *P<0.05. **P<0.01. ***P<0.001.

FIG. 7, is a set of graphs showing peripheral Blood Mononuclear cells treated with escalating doses of FasL following different treatments. 2 h incubation with FasL-monunuclear cells were incubated for 2 hours with FasL at different concentrations. 2 h incubation with FasL+48 h activation-mononuclear cells were incubated for 2 hours with FasL at different concentrations and then activated for 48 hours with anti CD3 and anti CD28 antibodies. 48 h activation+2 h incubation with FasL-mononuclear cells were activated with anti CD3 and anti CD28 antibodies for 48 hrs and then incubated for 2 h with FasL. (A) Viable CD3⁺ cells following the different treatments, (B) Activated CD3⁺ cells expressing CD25⁺ following the different treatments (% CD3⁺/CD25⁺), (C) Early apoptosis of CD3⁺ cells (% CD3⁺/AnnexinV⁺).

FIG. 8, is a graph showing the effect of Fas-L on transduction efficiency and on the survival of transduced T-cells as measured by the percent of viable GFP⁺ cells of the total CD3⁺ cell population. Two concentrations of Fas-L were examined 50 ng/ml and 100 ng/ml and compared with no Fas-L (Ong/ml), at three different cell groups: one received Fas-L before activation, one received Fas-L after activation and one received Fas-L after transduction. Standard CAR-T are cells treated per the standard procedures of CAR-T cells manufacturing.

FIG. 9, is a graph showing the transduction efficiency as measured by IFN-γ secretion (pg/ml) by ErbB2-CAR-T cells stimulated by exposure to their antigen MDA-MB-231 cells and GFP⁺ expression. 1—Before activation Fas-L 0 ng/ml; 2—Before activation Fas-L 50 ng/ml; 3—Before activation Fas-L 100 ng/ml; 4—After activation Fas-L 0 ng/ml: 5—After activation Fas-L 50 ng/ml: 6—After activation Fas-L 100 ng/ml; 7—After transduction Fas-L 0 ng/ml: 8—Standard CAR-T: UT—control untreated cells.

FIG. 10, is a set of graphs showing the effect of Fas-L treatment post transduction at concentrations of 1, 10, and 50 ng/ml on the number of CAR-T cells, as measured by the % of viable GFP⁺ cells of the total CD3⁺ cell population (A) and their activation state, as measured by the % of viable GFP⁺CD25⁺ cells of the cell population (B). The graphs compare the results in CD3⁺ cells, CD8⁺ cells and CD4⁺ cells.

FIG. 11, is a set of graphs showing the effect of escalating concentrations of Fas-L (0, 1, 10, 50 ng/ml) added post transduction on CD4⁺ and CD8⁺ T cell subtypes naïve, central memory (CM), effector memory (EM) and effector (eff) cells. (A) the composition of viable CD8⁺ transduced cells (GFP⁺ CD8⁺) subtypes; (B) Viable CD8⁺ To cells; (C) the composition of viable CD4⁺ transduced cells (GFP⁺ CD4⁺) subtypes. (D) Viable CD4⁺ T_{H} subtypes T_{H}1 and T_{H}17. All cells were analyzed at the end of the CAR-T production process, after Fas-L treatment and 4 days recovery with IL-2. Results are presented as mean+SD of a duplicate.

### DETAILED DESCRIPTION OF EMBODIMENTS

The present invention is based on the surprising finding that exposure of a heterogeneous population of immune cells, e.g. cells obtained from G-CSF mobilized peripheral blood samples of human donors, to the apoptosis-inducing ligand Fas-L, causes a shift in the composition and activation state of cells present in the sample. In particular, the cells that are affected by the treatment are mature, apoptosis susceptible T cell subtypes. B cells and myeloid cells, all of which express variable levels of the Fas (CD95) receptor.

Apoptosis is a programmed cell death, which may be mediated by specific receptors for members of the TNF superfamily (including for example FasL (the terms FasL and Fas-L are used interchangeably herein), TNFα, TRAIL. TWEAK). These receptors are expressed on a variety of cell populations, mostly on mature activated cells, in which the expression of these specific receptors is correlated with controlled cell death, making them apoptosis susceptible cells, while naïve cells are insensitive. Other cell types may be resistant to death ligand-induced apoptosis, despite death ligand receptor expression, due to intracellular mechanisms (Kim et al 2002). The differential sensitivity to induced cell death may be used as a selection tool.

Mature T and B cells express the Fas receptor and are susceptible to the apoptotic effects of Fas ligand (Sprent and Tough, 2001; Strasser et al 2009). The Fas-L treatment as proposed in the present invention uses this Fas-Fas ligand mechanism to eliminate these apoptosis susceptible, reactive cells, that are found in lower levels at a steady state in the blood of healthy donors, as well as in high levels in the blood of auto-immune patients or patients with inflammatory diseases, and thereby may reduce the acute, undesired, pro-inflammatory reaction.

The inventors of the present invention demonstrate that amongst the T cells in G-CSF mobilized peripheral blood cells, helper T cells (T_{H}) (i.e. CD4⁺ cells) express higher levels of Fas receptor (FasR) than cytotoxic T cells (TC) (i.e. CD8⁺), and that mature subtypes of both T_{H} and T_{C} cells (including memory and effector T cells, and T_{H}1/T_{C}1 and T_{H}17 cells), as well as T stem cell memory (T_{SCM}) cells express extensive levels of FasR as compared to naïve T cells.

Furthermore, the inventors show that in G-CSF mobilized peripheral blood cells that were incubated with an apoptotic inducer (e.g. FasL), a significant reduction of both CD4⁺ T_{H} cells and CD8⁺ TC cells occurred. Furthermore, FasL selectively depleted specific subtypes of both T_{H} and T_{C} cells, namely helper and cytotoxic T_{SCM} populations.

The naïve T cells derived T_{SCM} cells are a specific subtype of naïve T cells. Current studies indicate that upon activation, the T_{SCM} further differentiate into memory and effector T cells that play a significant role in T cell reconstitution and pro-inflammatory responses (Zhang et al 2005, and Roberto et al 2015). The T_{SCM} subtype was shown by the inventors to express high levels of FasR and thereby are the fraction of naïve population which is mostly susceptible to Fas-L treatment.

In addition to T cells, other immune cells such as B cells and myeloid cells are also affected by FasL treatment.

Accordingly, the present invention provides a method of modifying a mixed cell population such as an immune cell population, to comprise less differentiated immune cells (e.g. T cells, B cells and myeloid cells), by exposing the immune cell population to an apoptosis inducing ligand. Such a modified immune cell population can be used in any method comprising immune cell transplantation in which the elimination of apoptosis susceptible cells from the transplant may increase the utility of the transplantation by reducing pro-inflammatory reaction of the apoptosis susceptible cells, e.g. T or B or myeloid cells.

Therefore, in a first of its aspects, the present invention provides a method for producing a population of cells enriched with non-activated/non-mature cells, comprising:
a. obtaining a biological sample comprising a heterogeneous population of mammalian cells; and
b. contacting the obtained heterogeneous population of mammalian cells with an apoptosis inducing ligand in a container, wherein said contacting induces apoptosis of active/mature cells while non active/mature cells remain resistant to the apoptotic signal, thereby isolating a population of cells enriched for non-active/non-mature cells.

In one embodiment, said heterogeneous population of mammalian cells is a population of immune cells. Said heterogeneous population comprises apoptosis resistant and apoptosis susceptible immune cells, including apoptosis susceptible-T cells and/or apoptosis susceptible B cells.

As used herein the term “apoptosis susceptible-T cells” encompasses CD95⁺ T cell subtypes, including, but not limited to T_{H}1/T_{C1}. T_{H}17, T_{SCM}, T_{CM}, T_{EM}, and T_{eff}. In certain embodiments, these T cell subtypes are defined by the expression profile of certain markers, as follows:

T_{SCM} (CCR7⁺CD45RA⁺CD95⁺LFA1^{high}),

T_{CM} (CCR7⁺CD45RA⁻ CD95⁺LFA1^{high}),

T_{EM} (CCR7⁻CD45RA⁻ CD95⁺LFA1^{high}),

T_{eff} (CCR7⁻CD45RA⁺CD95⁺LFA1^{high}),

T_{H}1/T_{C}1 (CD3⁺ CD4⁺CXCR3⁺),

T_{H}17 (CD3⁺ CD4⁺ CCR6⁺CXCR3⁻).

As used herein the term “naïve T cells” encompasses cells that are CD95⁻. In one embodiment naïve T cells are defined by the following expression profile: CCR7⁺CD45RA⁺CD95⁻LFA1^{low}.

As used herein the term “apoptosis susceptible B cells” encompasses CD95⁺ B cell subtypes, including, but not limited to Plasma blast, memory cells, transitional or naïve B cells. In certain embodiments, these B cell subtypes are defined by the expression profile of certain markers, as follows:

B transitional (CD27⁻CD38⁺)

B naïve (CD27⁻CD38⁻)

B Memory cells (CD27⁺CD38⁻)

B Plasmablast (CD27⁺CD38⁺)

In one embodiment, said container is made of a biocompatible material. In one embodiment, said apoptosis-inducing ligand is immobilized to an inner surface of the container.

According to another embodiment, said apoptosis-inducing ligand is immobilized to the surface of beads present within the container.

According to another embodiment, the container is selected from a group consisting of a bag, a column, a tube, a bottle, a vial and a flask.

In one embodiment, the apoptosis inducing ligand is selected from the group consisting of TNF-α, Fas ligand (FasL), TRAIL and TWEAK.

In a specific embodiment, the apoptosis inducing ligand is Fas-L.

The existing technologies of adoptive cell therapies use modified, activated or engineered autologous cells. One of the limitations of the autologous based therapies, is the need to generate tumor specific lymphocytes for each individual patient, which is technically and economically challenging. However, allogeneic adoptive transfer faces the danger of graft-versus-host-disease (GvHD). Pre-selection of the administered activated T cells, to reduce the GvHD causing cells, could result in a tumor specific treatment, without the risk of off-tumor damage.

Therefore, in one embodiment, the method of the invention can be employed in the preparation of autologous cell populations expressing a recombinant B cell antigen receptor, e.g. CAR-T cell transplantation, while reducing the risk of high levels of released cytokines.

In another embodiment the method of the invention can be employed in the preparation of allogeneic cell populations expressing a recombinant B cell antigen receptor, e.g. CAR-T cell transplantation, while reducing the risk of high level release of cytokines and in addition mitigating the risk of GvHD.

In another embodiment the method of the invention can be employed for reducing inflammatory causing cells with auto reactivity, such as in T cell mediated autoimmune and inflammatory diseases, including but not limited to Multiple Sclerosis (MS), Rheumatoid Arthritis (RA), Autoimmune Diabetes, Diabetes mellitus type 1 and type 2, SLE (Systemic Lupus Erythematosus), Myestenia gravis, Progressive systemic sclerosis, Hashimoto's thyroiditis, Grave's disease. Autoimmune haemolytic anemia. Primary biliary cirrhosis, Crohn's disease, Ulcerative Colitis, Rheumatoid Spondylitis, Osteoarthritis. Gouty Arthritis, Arthritic conditions, Inflamed joints, Eczema, Inflammatory skin conditions, Inflammatory eye conditions, Conjunctivitis, Pyresis, Tissue necrosis resulting from inflammation, Atopic dermatitis, Hepatitis B antigen negative chronic active hepatitis, Airway inflammation, Asthma and Bronchitis.

In one embodiment, the method of the invention can be employed for decreasing immunological activity by reducing the pro-inflammatory T_{H}1 and T_{H}17 populations, which are known to elevate autoimmune reactions in autoimmune Multiple Sclerosis (MS) (Baecher-Allan et al, 2018). Namely, in accordance with one embodiment of the invention, the MS patient's peripheral mononuclear cells are removed temporarily, treated with an apoptosis-inducing ligand (e.g. FasL), resulting in lowering the autoimmune load and re-transplanted into the patient clean from autoreactive clones.

In another embodiment the method of the invention can be employed for reducing auto-antibody producing B cells or B cell antigen presentation, in autoimmune diseases such as, but not limited to, Lupus erythematosus (Nashi et al, 2010), Multiple Sclerosis (Baker et al, 2017).

In one embodiment the method of the invention can be employed for using progenitor cells such as Multipotential Stromal/Mesenchymal Stem Cells, Neural Progenitor Cells and Endothelial Progenitor Cells in regenerative medicine, in improving the outcome due to administration of a selected population.

In another embodiment, the method of invention can be employed in facilitating the use of double cord blood as a method for hematopoietic stem cell transplantation, namely, in lowering the GvHD and the cross attack of one cord unit's cells to the other.

In another embodiment, a heterogeneous population of donor cells is obtained (e.g. G-CSF (Granulocyte Colony Stimulating Factor) Mobilized Peripheral Blood cells obtained from apheresis of healthy, consenting, stem cell donors). The cells are incubated with an apoptosis inducing ligand (e.g. Fas Ligand). FasL is removed from the cell culture, e.g. by one or more washing steps. In one embodiment, no further isolation steps are performed.

In certain embodiments, incubation with the apoptosis-inducing ligand (e.g. FasL) may be performed in a device having FasL attached to a surface thereof.

The present invention discloses a method for producing a cell population from which specific subtypes of apoptosis susceptible cells are depleted. The method enables simultaneous positive selection for immune cells which support engraftment, the desired activity such as anti-tumor activity, cells which support tissue regeneration and negative selection for cells which have a detrimental effect such as release of life threatening levels of cytokines, cells which are directed to self-antigens, cells which are the key players in causing graft versus host disease (GvHD), cells which have an inflammatory causing profile or other effects, out of a heterogeneous cell population.

The immune cell population comprises apoptosis-signaling resistant cells and apoptosis-signaling sensitive cells. The method comprises providing a sample comprising a heterogeneous cell population, incubating the cells with an apoptosis inducing ligand, thereby eliminating the more apoptosis-sensitive cells (e.g. mature effector cells) from the sample and enriching the population with the apoptosis-signaling resistant cells (e.g. naïve-T or B or myeloid or CD34 cells or other progenitors).

Described are methods for preparing populations of cells, such as genetically modified T cells, e.g. T cells expressing a chimeric antigen receptor, or some other activated T cells and having lower toxicity and GvHD or other toxic activity. The method entails contacting the cells with an apoptosis inducing ligand, e.g., during various steps of the therapeutic cell preparation, for example prior to or after culturing and expansion of the T cell population expressing the recombinant antigen receptor.

A chimeric antigen receptor (CAR) is a recombinant biomolecule that can bind specifically to a target molecule present on the cell surface of a target cell, for example, the CD19 antigen on B cells. Non-limiting examples of CAR molecules include a chimeric T-cell receptor, an artificial T-cell receptor or a genetically engineered receptor. These receptors can be used to endow the specificity of a monoclonal antibody or a binding portion thereof onto a desired cell, e.g. a T cell. CARs can bind antigen and transduce T cell activation, independent of MHC restriction. Thus, CARs are “universal” immune-receptors which can treat a population of patients with antigen-positive tumors irrespective of their HLA genotype. Adoptive immunotherapy using T lymphocytes that express a tumor-specific CAR can be a powerful therapeutic strategy for the treatment of cancer.

CAR coding sequences can be produced by any means known in the art, though preferably it is produced using recombinant DNA techniques. Nucleic acids encoding the several regions of the chimeric receptor can be prepared and assembled into a complete coding sequence by standard techniques of molecular cloning known in the art (genomic library screening, PCR, primer-assisted ligation, site-directed mutagenesis, etc.). The resulting coding region is preferably inserted into an expression vector and used to transform a suitable expression host cell, preferably a T lymphocyte. There are several available techniques for inserting a gene into a host genome, using viral or non-viral transfection vectors. For example, a nucleic acid may be injected through a cell's nuclear envelope directly into the nucleus or administered to a cell using viral vectors to produce genetically modified cells.

Transfection with a viral vector is a common technique for producing genetically modified cells, such as T cells. This technique is known as viral transduction. The nucleic acid is introduced into the cells using a virus, such as a lentivirus or adenovirus, or a plasmid, as a carrier using methods well known in the art.

Peripheral blood mononuclear cells as well as enriched T cell populations (e.g. CD4+ and CD8+ T cells) can be isolated by various methods, transduced with a vector for CAR expression and cultured by the methods described herein.

As used herein the term “CAR-T” or “CAR-T cells” refers to T cells that were transduced with a CAR construct.

As used herein the term “CAR construct” refers to a vector comprising the gene encoding the desired CAR, optionally further comprising additional nucleic acid sequences required for expression of said gene and optionally further comprising additional components encoding accessory molecules for enhancing the CAR function.

As used herein the term “mononuclear cells” refers to any blood cell having a round nucleus. These cells consist of lymphocytes (T cells, B cells, NK cells) and monocytes. The term “peripheral blood mononuclear cells” refers to a mononuclear cell found in peripheral blood.

PBMC can be isolated from whole blood using methods well known in the art, for example using ficoll, a hydrophilic polysaccharide that separates layers of blood, and gradient centrifugation, which will separate the blood into a top layer of plasma with platelets, followed by a layer of mononuclear cells and a bottom fraction of polymorphonuclear cells (such as neutrophils and cosinophils) and erythrocytes.

For example, T cells can be isolated from peripheral blood by gradient separation, elutriation or affinity purification. The cells are incubated with an apoptosis-inducing ligand and thereby the cell population is shifted towards a more immature state. The cells can then be transduced with, for example, a SIN lentiviral vector that directs the expression of a CAR (e.g., a CD19 or HER2 specific CAR). The genetically modified T cells can be expanded in vitro and then cryopreserved or provided freshly for immediate use. Alternatively, the T cells can be transduced with, for example, a SIN lentiviral vector that directs the expression of a CAR (e.g., a CD19 or HER2 specific CAR), then the cells are incubated with an apoptosis-inducing ligand and thereby the cell population is shifted towards a more immature state. The selected, genetically modified T cells can be expanded in vitro and then cryopreserved or provided freshly for immediate use.

As demonstrated in the Examples below, exposure of peripheral blood mononuclear cells to FasL prior to activation with anti-CD3/CD28 antibodies resulted in selection for cells with higher potential to be efficiently transduced into CAR-T cells, as measured by the number of CAR expressing cells and by the level of IFNγ secreted by these cells upon exposure to the target antigen.

Therefore a step of exposure to FasL during the procedure of CAR-T production (and in particular prior to the activation step of the cells) may result in improved transduction, in particular, but not limited to, in the setting of autologous CAR-T transplantation, where transduction efficiency is impaired, for example due to previous chemotherapy treatments.

In addition, as demonstrated in the Examples below, FasL treatment after transduction may decrease potential pro inflammatory CAR T-cells and their activation state. Therefore, a step of exposure to FasL after the transduction step may result in reducing the cytokine release storm, or mitigating GvHD development in the setting of allogeneic CAR-T transplantation.

Therefore, in another one of its aspects the present invention provides a method for producing CAR-T cells, said method comprising:
a. Isolating mononuclear cells from a biological sample;
b. Activating the cells by contacting said cells with a T cell activating agent (e.g. anti-CD3/CD28 antibodies);
c. Transducing said cells with a CAR construct, wherein said method further comprises contacting said cells with an apoptosis inducing ligand before the activating step (b) and/or after the transducing step (c), thereby obtaining CAR-T cells.

In certain embodiments said method results in obtaining improved transduction efficiency. In certain embodiments said method results in reduced cytokine release storm, or reduced GvHD in the patient, in the setting of allogeneic CAR-T transplantation.

In one embodiment, said isolated mononuclear cells are peripheral blood mononuclear cells. In some embodiments said mononuclear cells are enriched with CD3⁺, CD4⁺ and/or CD8⁺ T cells.

In one embodiment, said activating step (b) is performed for a period of between about 1-3 days. In one specific embodiment said activating step is performed for about 48 hours (2 days).

The terms “Transduction” or “Transducing” as used herein refer to methods of transferring the CAR construct into the T cell by way of a vector which results in integration of the CAR transcript into the cell. Common techniques use infection with a virus, viral vectors, electroporation, protoplast fusion, transposon/transposase system (e.g. see Hackett et al (2010)), and chemical reagents to increase cell permeability, e.g. calcium phosphate transfection. Viruses commonly used for gene therapy are adenoviruses, adeno-associated viruses (AAV), retroviruses or lentiviruses, for example.

Terms in the disclosure herein should be given their plane and ordinary meaning when read in light of the specification. One of skill in the art would understand the terms as used in view of the whole specification.

As used herein, “a” or “an” may mean one or more than one.

As used herein, the term “about” indicates that a value includes the inherent variation of error, e.g. a 10% variation.

### EXAMPLES

### Example 1: FasL Treatment has a Differential Effect on Different T Cell Subtypes

This experiment was performed with samples of G-CSF (Granulocyte Colony Stimulating Factor) Mobilized Peripheral Blood cells (MPBC) obtained from apheresis of healthy, consenting, stem cell donors. Donors received G-CSF (10-12 μg/kg/day) for a period of 4-5 days prior to the leukapheresis. The cells underwent two washing steps with buffer containing EDTA, and were incubated at a concentration of 100±20×10⁶ cells/ml in CellGro SCGM medium (CellGenix) with recombinant human Fas Ligand (Mega FasL, Adipogen) at a concentration of 100 ng/ml for 2 hours at 37° C. in a humidified incubator 5% CO₂. Following the incubation with FasL, the cells were subjected to two additional washing steps to remove unbound FasL. No further isolation steps were performed. Control non treated samples consisted of the original unprocessed MPBC from the same donor.

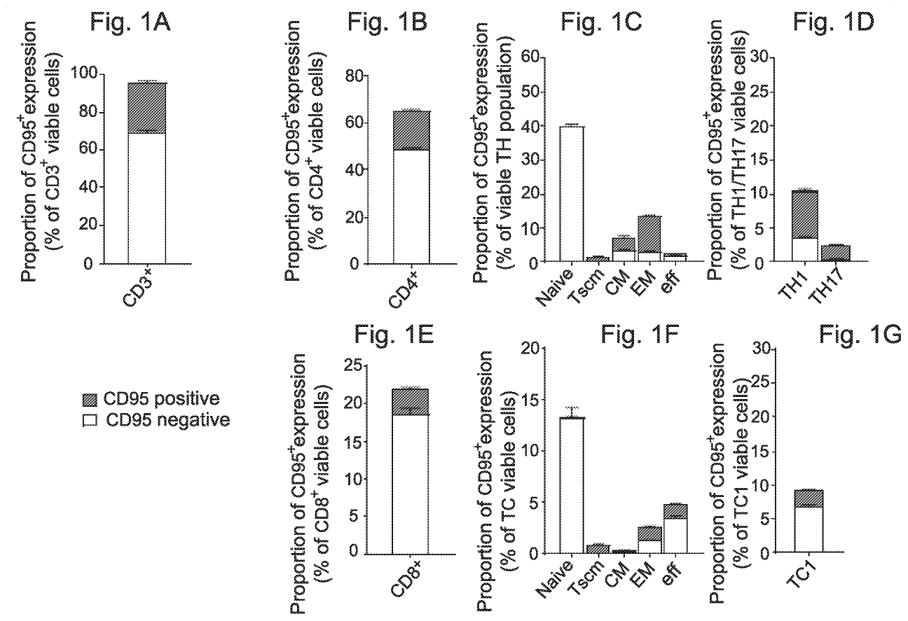

Immunophenotyping of the T cell subtypes was performed by flow cytometry using the following antibodies (Miltenyi): CD4, CD8, CCR7, CD45RA, LFA1. CD95. CXCR3 and CCR6. Data from samples was acquired using flow cytometer (MACSquant, Miltenyi) (FIG. 1). The following populations were determined according to their receptor expression: T helper (T_{H}. CD4⁺), T cytotoxic (T_{C}, CD8⁺), their subtypes: Naïve T cells (CCR7⁺CD45RA⁺CD95-LFA1^{low}), T_{SCM} (CCR7⁺CD45RA⁺CD95⁺LFA1^{high}), T_{CM} (CCR7⁺CD45RA⁻), T_{EM} (CCR7⁻CD45RA⁻) T_{eff} (CCR7⁻CD45RA⁺), T_{H}1/T_{C}1 (CXCR3⁺), T_{H}17 (CCR6⁺CXCR3⁻). The expression level of FasR (CD95) on the surface of these T cell subtypes was analyzed.

The FasR (CD95) expression profile described in FIG. 1 reveals that helper T (T_{H}) cells (CD4⁺) express higher levels than cytotoxic T (T_{C}) cells (CD8⁺), and that mature subtypes of both T_{H} and T_{C} cells (including memory and effector T cells, and TH1/TC1 and T_{H}17 cells) as well as T_{SCM} cells express extensive levels of FasR as compared to naïve T cells.

### Example 2: Population Percentage and Apoptosis of T Cell Substrates

Samples of G-CSF MPBCs obtained from apheresis of healthy donors within 24 hours of collection, were incubated with or without Fas ligand, in a closed infusion bag system. Briefly, cells were counted, washed twice with buffer containing EDTA, incubated at a concentration of 100±20×10⁶ cells/ml in CellGro SCGM medium (CellGenix), in the presence of the apoptotic mediator Fas ligand (MegaFasL, Adipogen) at a concentration of 100 ng/ml for 2 hours at 37° C. in a humidified incubator 5% CO₂, then washed twice to remove unbound FasL. T cells were isolated from MPBCs after incubation with Fas ligand or control MPBC, using magnetic Human T cell isolation beads (EasySep, StemCell, 17951) according to the manufacturer's protocol. Immunophenotyping of the isolated T cell subtypes was performed by flow cytometry using the following Miltenyi Abs: CD4, CD8, CCR7, CD45RA, LFA1, CD95, CXCR3 and CCR6. Data from samples was acquired using flow cytometer (MACSquant, Miltenyi). The following populations were determined according to their receptor expression: T helper (T_{H}, CD4⁺), T cytotoxic (T_{C}, CD8⁺), Naïve T cells (CCR7⁺CD45RA⁺CD95-LFA1^{low}), T_{SCM} (CCR7⁺CD45RA⁺CD95⁺LFA1^{high}), T_{CM}(CCR7⁺CD45RA⁻), T_{EM}(CCR7⁻CD45RA⁻) and T_{eff} (CCR7⁻CD45RA⁺), T_{H}1/T_{C}1 (CXCR3⁺), T_{H}17(CCR6⁺CXCR3⁻). In addition, the apoptosis and necrosis levels of the T cell subtypes were assessed using Annexin V staining (eBiosciences BMS500FI) and 7AAD (eBiosciences 00-6993) staining, where Annexin V⁺7AAD⁻ cells were defined as early apoptotic, and all of the 7AAD⁺ cells were considered late apoptotic/necrotic cells, and were gated out of the analysis of the viable cells.

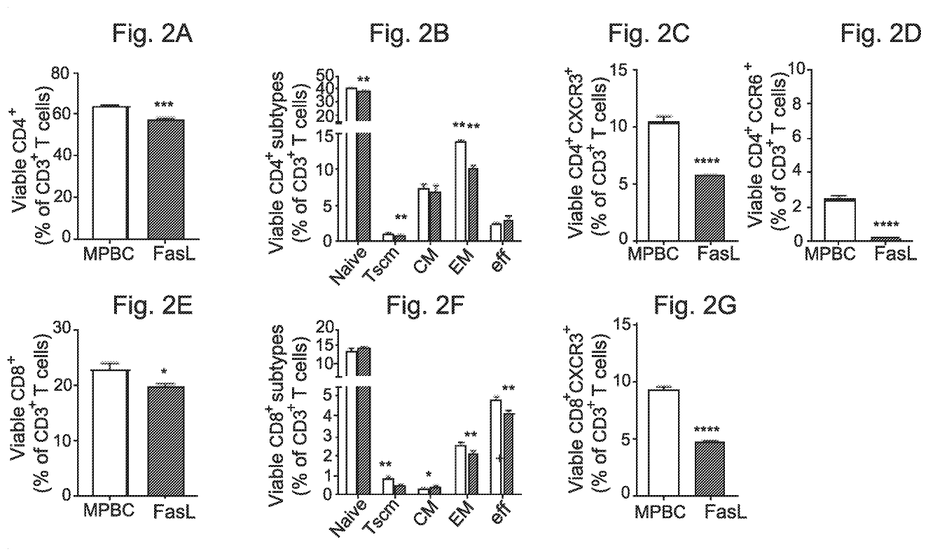

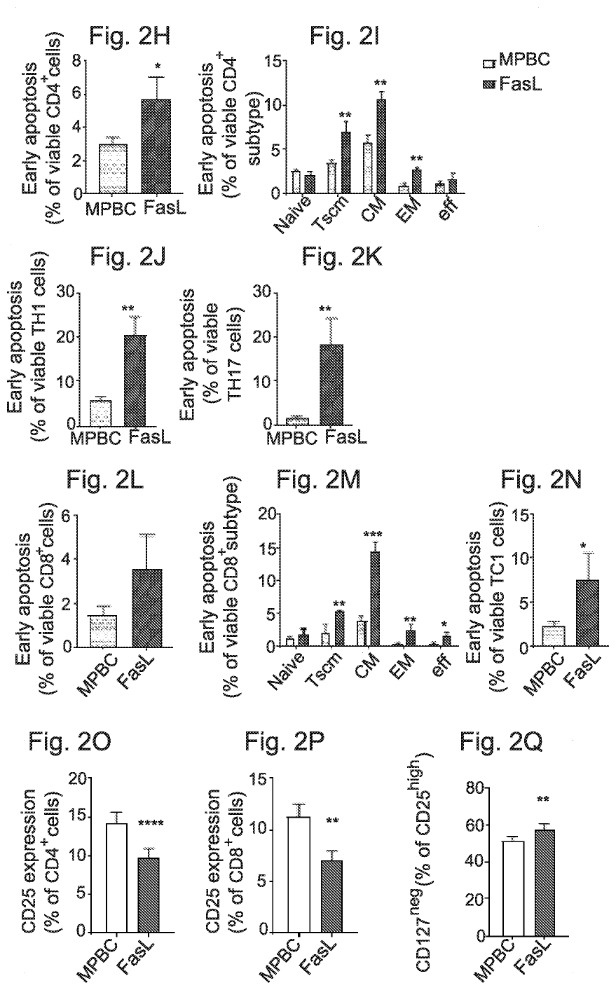

FasL treatment selectively depleted both helper and cytotoxic T cell subsets. As can be seen in FIG. 2A-2G, the percentage of helper and cytotoxic T_{SCM} and T_{EM} cells decreased upon incubation with FasL. In addition, the percentage of T_{H}17 and T_{H}1 and T_{C}1 cells decreased significantly as a result of incubation with FasL: FasL treatment preferentially induced apoptosis in T_{H}1, T_{C}1 and T_{H}17 populations (45%, 48% and 92%, respectively, P<0.0001) while the naïve-T_{H} and T_{C} cells were less affected.

As shown in FIG. 2A-2G and as will be elaborated further below. MPBCs which were incubated with the apoptotic inducer (FasL), showed a significant reduction of both CD4⁺ T_{H} cells (10.7%, P<0.001) and CD8⁺ T_{C} cells (14.0%, P<0.05). Furthermore, FasL selectively depleted specific subtypes of both T_{H} and T_{C} cells. The results provided herein further demonstrate a statistically significant reduction of helper (23.2% P<0.01) and cytotoxic (41.8%, P<0.01) T_{SCM} populations.

The results indicate that exposure to the apoptosis-inducing ligand Fas-L, selectively depletes T Helper (T_{H}) cells: memory-T_{H} cell subsets are reduced, while in the naïve-T_{H}, only a small portion of the cells express FasR (namely the T_{SCM} subset) and this specific subset is affected by the apoptotic challenge. Moreover, it appears that exposure to Fas-L preferentially induces apoptosis in pro-inflammatory T_{H}1, T_{C}1 and T_{H}17 populations (FIG. 2H-2N).

Without wishing to be bound by theory such results indicate that incubation with the apoptosis inducing ligand Fas-L results in elimination of the apoptosis susceptible matured helper T-cells, leaving a less differentiated state of the subpopulation.

In addition, pre-treatment with FasL also reduced significantly effector memory T helper (TH_{EM}) subtype as well as effector memory T cytotoxic (TC_{EM}) and effector (TC_{eff}) subtypes (26.0%, P<0.0001, 16.4%. P<0.01; 13.6%, P<0.01, respectively), while in the naïve T_{C} subtype there was no effect and in the naïve T_{H} cells, only a slight reduction was shown (6.0%, P<0.01). Furthermore, the level of pro-inflammatory mature T cells, T_{H}1, T_{C}1 and T_{H}17 was massively reduced (55.1%, 47.9% and 91.8% respectively, with P<0.0001) as compared to MPBCs control. In addition to the reduction in viable population percentages of each subtype of T cell, the early apoptosis level was shown to increase following FasL treatment. FIG. 2H-2N presents 1.93 fold increase of CD4 helper T (T_{H}) early apoptosis levels (P<0.05) as well as 2.48 fold increase of CD8 cytotoxic T cells (T_{C}) (P=0.08). In addition, the early apoptosis level is significantly elevated in the T_{SCM} (2.00 fold, P<0.01; 2.42 fold, P<0.01), CM (1.87 fold, P<0.01; 3.78 fold, P<0.001), and EM (2.89 fold, P<0.01; 6.09 fold, P<0.01) subtypes of both T_{H} and T_{C} respectively, while there was no change in the early apoptosis level of the naïve T cells as compared to MPBCs. Furthermore, the early apoptotic level of pro-inflammatory mature T cells, T_{H}1, T_{C}1 and T_{H}17 was significantly elevated (3.6 fold, P<0.01; 3.4 fold, P<0.05; and 11.4 fold, P<0.01 respectively) as compared to MPBC control. In FIG. 2O-2Q the percentage of helper and cytotoxic T cells expressing the CD25 activation marker is significantly reduced. CD25 receptor is known to be up-regulated during T cell activation. In addition, the proportion of regulatory T cells (Tregs) which are responsible for anti-inflammatory reaction, in the total CD25⁺ T helper cells showed significant elevation.

Overall these data suggests that unlike complete T cell depletion methods currently used, the pre-treatment with FasL selectively depleted specific sub-populations, and reduces activation. Therefore, and without wishing to be bound by theory, it appears that treatment with an apoptosis inducing ligand such as FasL results in elimination of apoptosis susceptible matured T-cells, leaving a less differentiated state, less activated and maintain anti-inflammatory T cells subpopulations.

### Example 3: Reduced Activation of Fas-L Pre-Treated T Cells Subtypes in Response to In-Vitro Activation

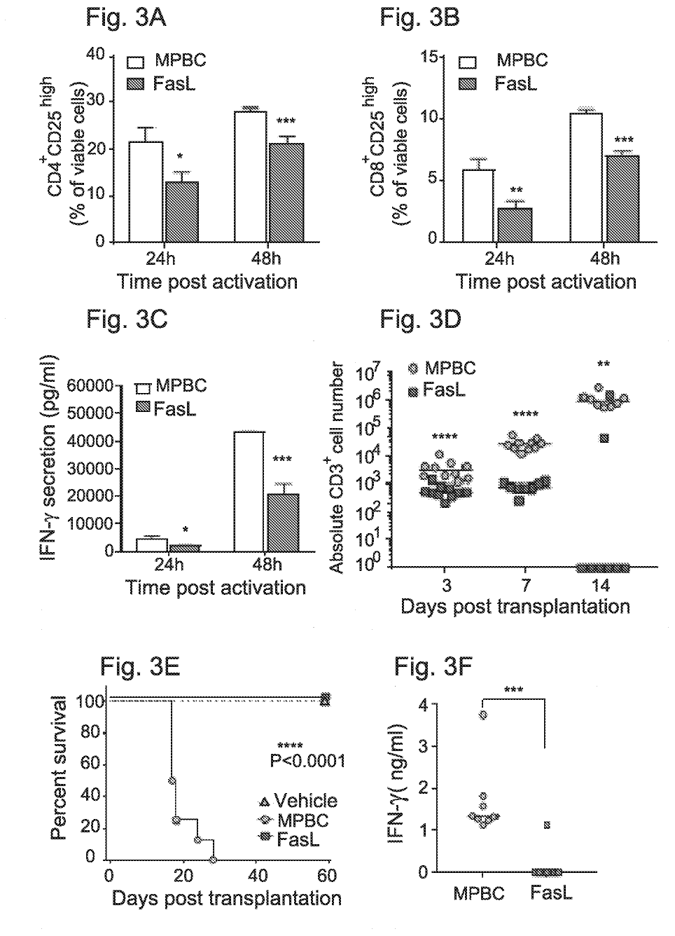

Next, the expression level of CD25 receptor, the marker for cell activation, was evaluated in activated T cell subtypes. T cells were isolated from FasL pre-treated MPBCs, and MPBC controls, and incubated 1 or 2 days with anti CD3/CD28 activation beads. As seen in FIG. 3A-3B, and in line with the above results, following day 1 and 2 of activation there was a significant reduction of CD25^{high} expressing FasL pre-treated CD4⁺ cells (39.7% and 24.3%: P<0.05 and P<0.001 respectively) and CD8⁺ cells (53.3% and 33.9%; P<0.01 and P<0.001 respectively), as compared to control T cells. Furthermore, the pro-inflammatory cytokine IFNγ secretion showed in FIG. 3C was significantly lower on days 1 and 2, following incubation (56.1% P<0.05, and 52.1%. P<0.001, respectively), indicating a less activated state of the FasL pre-treated T cells as compared to MPBC control T cells. The results of FIG. 3D-3F present reduced inflammation in GvHD mouse model. γ-irradiated IL2Rγ-null (NSG) mice were transplanted with Fas-L treated or control MPBCs. On days 3, 7 and 14 post transplantation there was reduced absolute cell number of CD3⁺ T lymphocytes in the spleens that were harvested from mice transplanted with FasL-treated-MPBCs (FIG. 3D). The progression of the GvHD was fatal in the MPBC transplanted group with no mice surviving beyond day 28 post transplantation. In contrast, transplantation of FasL treated MPBCs significantly prolonged mice survival (P<0.0001), as no animal was found dead during 60 days of follow-up (FIG. 3E) and there was no detection of IFNγ cytokine in the serum on day 14, compared to high levels of IFNγ detected in the serum of MPBC control mice (FIG. 3F). These results support the results shown in FIG. 2, in which, FasL treated MPBCs show, a less differentiated and less activated profile of T cells subtypes, leading to reduced activation following stimulation.

In these experiments, the isolated T cells from MPBC controls and MPBC incubated with Fas-L were counted and incubated at 0.75×10⁶ cell/ml in RPMI complete medium (supplemented with 10% FCS, 1% L-Glutamine, 1% Pen-Strep, 1% non-essential amino acid and 1% sodium pyruvate), and stimulated using activation beads (Dynabeads™ Human T-Activator CD3/CD28 Gibco 111.32D), at a 1:10 bead:cell ratio, for 24/48 hrs. For analysis of T cell subtypes, the cells were stained with all the Abs described above in Example 2. In addition, flow cytometry analysis was performed for CD25 activation receptor expression. Furthermore, IFNγ cytokine secretion using ELISA was also performed according to manufacturer's protocol (R&D systems, Quantikine ELISA kit DIF-50).

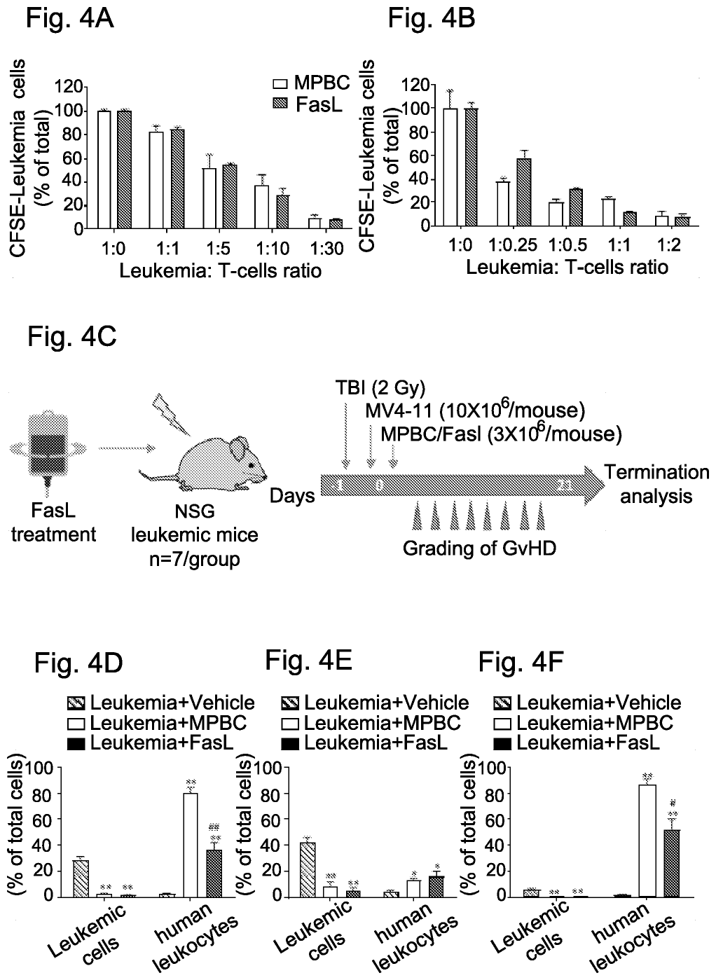

It is important to note that the elimination of apoptosis susceptible mature cells, as well as reduced activation state, as demonstrated in the data above, is not affecting other attributes of the T cell population. FIG. 4 reveals that FasL treatment of the MPBC, does not affect the Graft versus Leukemia activity (see details in Example 4 below).

### Example 4: FasL Treatment does not Affect Graft Versus Leukemia Cytotoxic Activity In-Vitro and In-Vivo

Fas-L treated MPBC or control cells were expanded by incubation in a 24 well-plate at concentration of 1×10\hat{ }6 cells/ml, in complete RPMI medium (containing 10% FCS, 1% L-Glutamine, 0.2% β-Mercaptoethanol, 1% Pen/Strep, 1% sodium pyruvate and 1% non-essential amino acids) and supplemented with 30 μg/ml anti-CD3 (eBioscience, 16-0037, OKT3) and 1000U/ml recombinant IL2 (hr-IL-2 R&D systems, 202IL-500). On the 4th day, the medium was replaced with complete fresh medium (containing anti CD3 and IL-2) and the cells were counted and re-seeded at 5×10\hat{ }6 cells/ml in 6-well plates. The cells were counted, and the medium was replaced every other day. On day 12 of the expansion, two different types of Leukemia cell lines—MV4-11 and U937 cells were labeled with 2 μM CFSE (eBioscience, 65-0850), and seeded in complete RPMI at 2×10\hat{ }4/100 μl in a 96-well plate.

The expanded Fas-L treated MPBC or control cells were washed, counted and co-cultured overnight in elevated concentrations with the labeled Leukemia cells (MPBC:leukemic cells ratio of 1:1, 1:5, 1:10 and 1:30). At the end of the incubation, cells were stained with Propidium Iodide for detection of dead cells, the number of viable CFSE-leukemic cells was analyzed using FACSCalibur Flow Cytometer (BD Biosciences, San Jose, Calif., USA); the data was analyzed using BD CellQuest software (version 3.3: BD Biosciences) (FIG. 4A-4B).

Furthermore, the ability of MPBCs to kill Leukemia cells was evaluated as well using in-vivo mouse model. In this model, MV4-11 leukemic cells were administered into γ-irradiated NSG mice on day 0 (10×10⁶ cells/mouse), and FasL-treated and control MPBC grafts (3×10⁶ total nucleated cells (TNCs)/mouse) were injected within 4-6 hours (FIG. 4C). Three weeks post transplantation in both mice groups the leukemic cells were similarly diminished from the spleen. BM, and blood of co-transplanted mice as compared to vehicle (P<0.01) (FIG. 4D-4F). In summary, FasL treated MPBCs and control-MPBCs transplanted mice exhibit identical Graft versus Leukemia activity whereas the FasL treated MPBCs display additionally a reduction in GvHD.

### Example 5: FasL Treatment Reduces APCs Activation

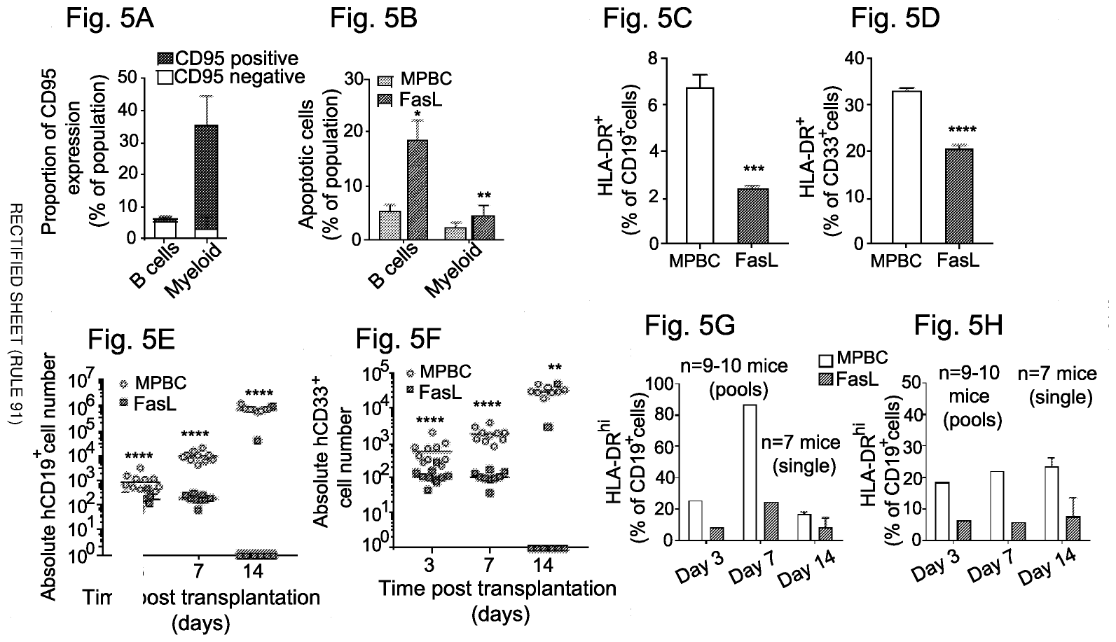

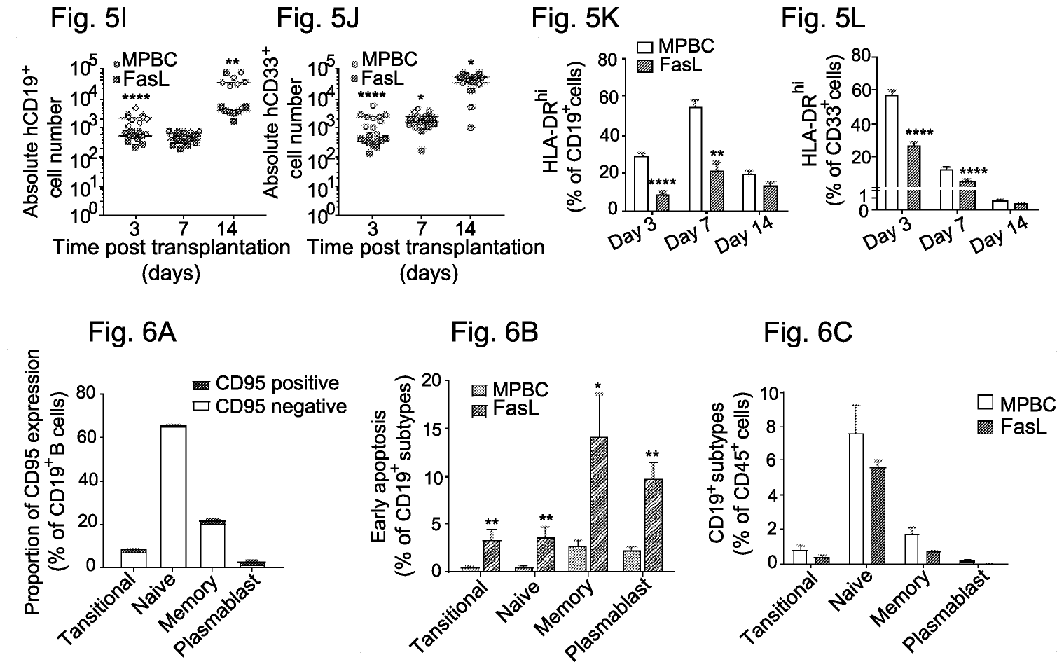

Alloreactivity of T cells depends, among others, on antigen presentation of myeloid cells (dendritic cells and monocytes) as well as B cells that serve as antigen presenting cells (MacDonald et al, 2013). In addition to the T cells population, the APCs express CD95 and are exposed as well to the FasL, therefore, we hypothesized that they may play a role and contribute to the reduction in GvHD. Assessment of CD95 expression in untreated MPBCs revealed moderate levels in the B cells population and high levels in the myeloid cells (FIG. 5A). A significant elevation of apoptotic cell percentage was detected in both B and myeloid cells in FasL treated MPBCs (FIG. 5B), which was associated with a significant 0.36 fold (P<0.001) and 0.62 fold (P<0.0001) reduction of HLA-DR, an MHC class II cell surface receptor, responsible for antigen presentation, in both cell populations, respectively (FIG. 5C-5D). FIG. 3D described the results of an in-vivo experiment showing significantly reduced human T cell number in the spleen of FasL treated MPBCs transplanted mice, at days 3, 7 and 14. A significantly low number of human B cells and human myeloid cells was further found in the spleen of these FasL treated MPBCs transplanted mice (FIG. 5E-5F), which expressed extremely low levels of the HLA-DR^{hi} antigen presentation mediator, indicating reduced activation levels of these cells (FIG. 5G-5H). Similar results were found as well in the Bone Marrow of these mice, transplanted with FasL treated MPBCs, showing significantly reduced numbers of human B and myeloid cells, and significantly lower levels of HLA-DR^{hi} expressing cells (FIG. 5I-5L).

### Example 6: B Cell Subtypes Express FasR and Respond to Apoptosis Induction

FasR (CD95⁺) expression of MPBC control cells was measured in B cells subtypes, using anti CD95 antibodies (Miltenyi). Analysis of the B cell subtypes was performed using the following antibodies: anti CD19, anti CD27 and anti CD38. Data from samples was acquired using flow cytometer (MACSquant, Miltenyi). The following B cells sub-populations were determined according to their receptor expression: Transitional (CD27⁻CD38⁺), naïve (CD27⁻CD38⁻), memory (CD27⁺CD38⁻), and plasmablast (CD27⁺CD38⁺). In addition, the early apoptosis of the B cell subtypes was assessed using Annexin V (eBiosciences BMS500FI) and 7AAD (eBiosciences 00-6993) staining, where Annexin V⁺7AAD⁻ cells were defined as early apoptotic, and all of the 7AAD⁺ cells were considered late apoptotic/necrotic cells, and gated out of the analysis of the viable cells.

FIG. 6 displays the FasR expression level (A), percentage of early apoptotic cells (B) and percentage of B cell subtypes (C) following 2 hours incubation with FasL and in control cells. It can be seen in FIG. 6A that the proportion of Plasmablast B cell subtype, which is the most mature subtype of B cells, and express FasR on their surface, is the highest compared to transitional/naïve cells, which are early differentiated B cells. Consistent with the high FasR expression, this population showed the strongest early apoptosis signal following incubation with FasL (FIG. 6B). Other B cell subtypes were also affected by the FasL treatment, as the percentages of early apoptotic cells were elevated, and the populations were reduced following FasL treatment (FIG. 6C), indicating that there are apoptosis susceptible B cells in all of the B cell subtypes mentioned above.

### Example 7: Human Mesenchymal Stem Cells (MSCs) Express FasR and Respond to Apoptosis Induction

Human MSCs are maintained in their naïve-undifferentiated state in medium and passaged once they reach confluence. To assess the effect of FasL on MSCs, the cells are plated at a density of 5×10⁺ cells/cm² in six-well plates and treated with different doses of FasL (from 1 to 50 ng/ml). On different days the cells are detached and counted using a hemocytometer or an automated cell counter. The culture supernatant is collected and assayed for secretion of angiogenic cytokines (e.g. bFGF, FGF2, HGF, IL-8, TIMP-1, TIMP-2 and VEGF) and pro-inflammatory cytokines/chemokines (IL-6, CCL2, CCL7 and CCL8).

### Example 8: The Effect of an In Vitro Treatment of Human MSCs with FasL on Mitigation of GvHD In-Vivo

Human MSCs grown in culture with or without FasL are tested for the mitigation of GvHD in-vivo. NOD.SCID IL2Rg^{null} (NSG) mice are subjected to total Body γ-Irradiation (TBI). GvHD is induced by administration of Mobilized Peripheral Blood Cells (MPBCs). FasL treated or untreated MSCs are administered by intravenous (IV) bolus injection 1 to 10 days later. Body weight changes as well as development of GvHD symptoms are assessed twice a week. The mice are followed until death or euthanization. Survival curves and median survival times are calculated for each treatment group.

### Example 9: Evaluation of FasL effect on CAR-T cells manufacturing Process and outcome

As shown in previous examples, following short incubation (e.g. 2 hours) of G-CSF mobilized peripheral blood cells with the apoptotic mediator Fas Ligand (FasL), the T cells composition was altered. FasL treatment reduced the percentage of mature and activated T cells, as expressed by decreased percentage of effector and increased percentages of naïve cells. Moreover, following exposure to FasL, the proportion of active cells in the population was reduced (as noticed by the number of T cells expressing CD25 marker). In addition, upon in-vitro activation of FasL-treated-T-cells using anti CD3/CD28 beads, lower number of CD25 expressing T cells was detected, as well as reduced level of IFNγ secretion and reduced differentiation kinetics rate, indicating a less mature and active T cell composition.

Therefore, the following example was undertaken in order to test whether combining FasL in CAR-T cells manufacturing process, may reduce the potential cytokine release syndrome (CRS), improve chimeric antigen receptor transduction efficiency and maintain or even contribute to CAR-T survival and antitumor activity.

### I. Testing the Effect of Escalating FasL Concentrations on PBMCs Before and after T Cell Activation

The following experiment was a preliminary study intended for determination of FasL concentration range, for induction of T cells apoptosis, differentiation and effect on activation potential, in a Peripheral Blood Mononuclear Cells (PBMC) sample.

Peripheral blood mononuclear cells (PBMC) were separated from buffy coat on Ficoll gradient. The PBMC were treated with an escalating dose of FasL, before or after activation of the cells. Activation was performed in 24 well dishes coated with anti-CD3/CD28 antibodies. The FasL concentrations that were examined were 0, 1, 5, 10, 25, 50 or 100 ng/ml FasL.

Three groups of cells were analyzed:
1) Cells incubated for 2 h with FasL (MegaFasL Adipogen) at concentrations of 1-100 ng/ml.
2) Cells incubated for 2 h with FasL (MegaFasL Adipogen) at concentrations of 1-100 ng/ml then subjected to 48 h of activation with anti-CD3/CD28 antibodies (2 h incubation with FasL+48 h activation).
3) Cells that were initially activated for 48 h with anti-CD3/CD28 antibodies and then incubated with FasL for 2 hours (48 h activation+2 h incubation FasL).

Next, each group of cells was analyzed using flow cytometry.

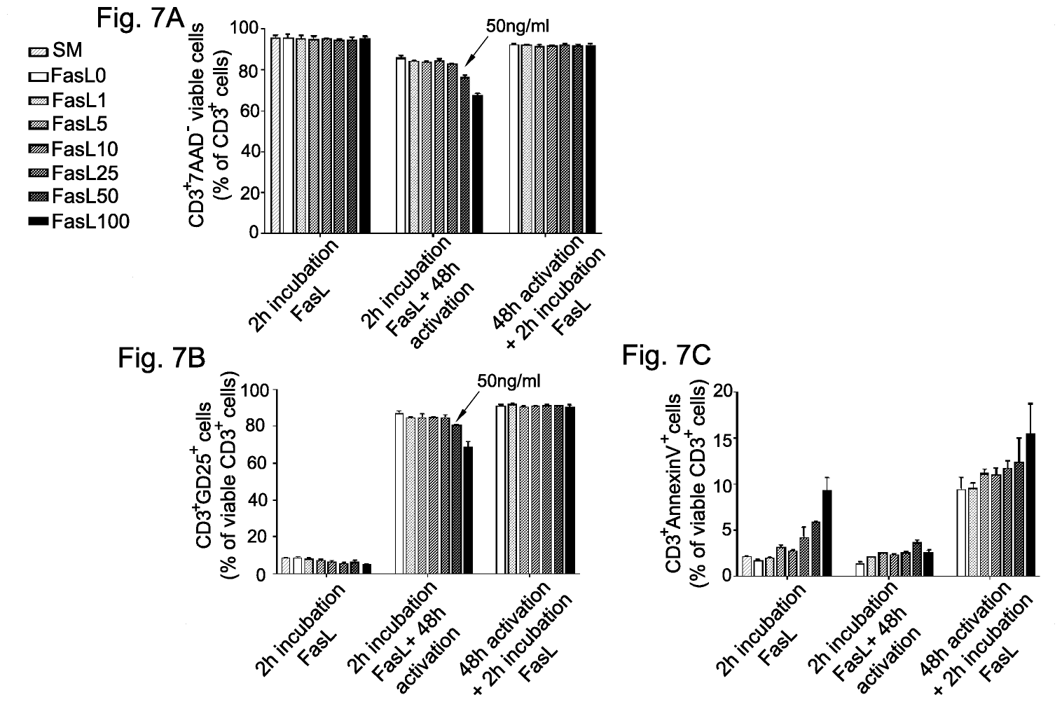

FasL effect on T cells Viability: significant reduction in the percentage of viable T cells (CD3⁺7AAD⁻ cells) was detected in T-cells treated with FasL at concentrations of 50 and 100 ng/ml followed by 48 h of incubation in activation conditions (with anti CD3/CD28 antibodies) (Group 2, FIG. 7A).

The effect of FasL on the activation state of T cells: the percentage of CD25 expressing T cells was reduced significantly with escalating concentrations of FasL in Group 2, but barely in Group 1 and none in Group 3, mostly in concentration higher than 25 ng/ml (FIG. 7B).

The effect of FasL on early apoptosis induction: Dose dependent elevation in early apoptosis levels (AnnexinV⁺ T cells) following treatment with escalating concentrations of FasL was detected when flow cytometry analysis was performed soon after FasL treatment (Groups 1 and 3, FIG. 7C). Following additional 48 h incubation in activation conditions, the early apoptosis levels were significantly lower (Group 2, FIG. 7C).

Based on these results, a concentration-range of FasL was delineated.

### II. Testing the Effect of FasL at Different Stages During the CAR-T Cells Manufacturing Process

PBMC were separated from buffy coats on Ficoll gradient. Activation was performed in 24 well dishes coated with anti-CD3/CD28 antibodies. Cells were treated with FasL at different stages during the CAR-T manufacturing process: before activation (Group 1), after activation (Group 2), and after CAR-T transduction (Group 3), in this case with ErbB2 CAR. FasL was used at concentrations of 0, 50 and 100 ng/ml. CAR-T transduction was performed with a lentivirus vector according to standard procedures (see for example Zhang et al 2017 Biomark. Res. 5:22: Fesnak et al Nature Protocols, Stem cell Technologies “Production of chimeric antigen receptor T cells”).

At the end of the CAR transduction process (following 10 days of incubation) the following parameters were evaluated: viability (7AAD⁻ cells), efficacy of CAR transduction (detected by elevation in the percent of GFP⁺ cells), differentiation state as indicated by the T cell subtypes (naïve/CM/EM/eff cells), and activation state (CD25⁺) were analyzed by flow cytometry.

In addition, a specificity assay was performed by incubation of T cells of each treatment group with the target antigen (human tumor cell line: MDA-MB-231). The ErbB2-CAR-T cells recognize the tumor cells and a pro-inflammatory reaction is initiated during which the cells release IFNγ into the medium. The media were collected and the level of IFNγ was evaluated using ELISA.

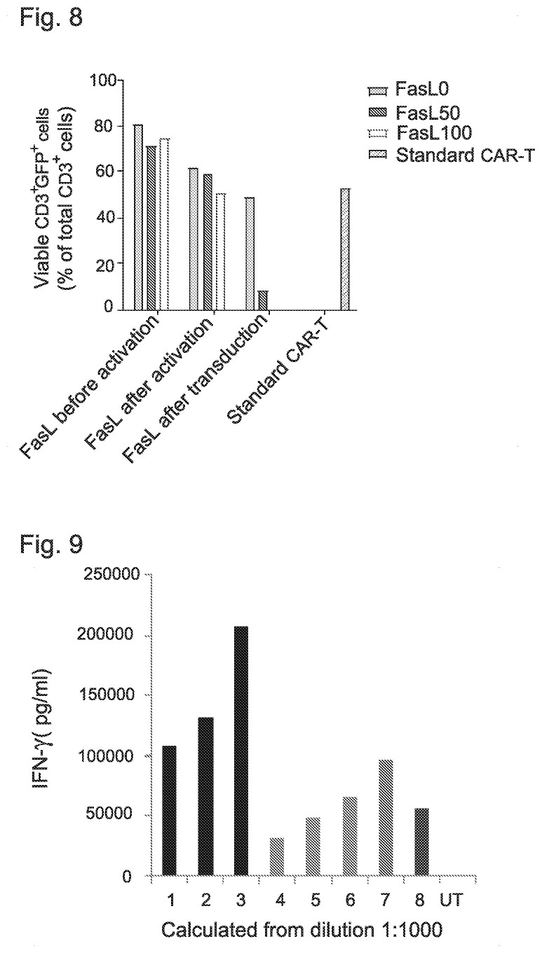

The results of this experiment demonstrated that: Exposure of cells to FasL prior to activation, resulted in improved CAR transduction in comparison to the standard CAR-T (FIG. 8). In this experiment the Ong/ml FasL (no FasL) sample gave high background of transduction.

Exposure to high concentrations of FasL (50-100 ng/ml) following CAR transduction, resulted in elevated cell death (decrease in viable transduced cells) in the cells that were incubated with 50 ng/ml FasL. No cells survived following treatment after transduction with 100 ng/ml FasL (FIG. 8).

Similar findings were obtained for CD4⁺ and CD8⁺ cells.

Following transduction of the ErbB2-CAR-T cells, the cells were co-cultured with their target tumour cells (the MDA-MB-231 human cell line). Cells that were exposed to FasL before activation, secreted high levels of IFNγ (FIG. 9) in comparison to the standard CAR-T and to cells exposed to FasL after activation. The elevated secretion of INFγ seems to correlate with elevated concentration of FasL (FIG. 9). The efficacy of transduction as measured by GFP⁺ staining (FIG. 8) was in correlation with INFγ secretion.

To summarize the above results, FasL treatment before activation results in better CAR transduction (FIG. 8). Results show higher GFP⁺ cell percentage in this group as compared to STANDARD CAR-T.

The improvement in CAR transduction is also reflected in the assay that measured the stimulation of the CAR-T cells with their target tumor cells. Cells that were exposed to FasL before activation, and incubated with their target cells, secreted high levels of IFNγ (FIG. 9) in comparison to the standard CAR-T and to cells exposed to FasL after activation. The elevated secretion of INFγ seems to correlate with elevated concentration of FasL (FIG. 9).

These results point to a beneficial role that incubation with FasL before the activation step, may have on the transduction efficiency of CAR-T cells, and on the increased response of CAR-T cells to antigen.

### III. Testing the Effect of Low FasL Concentrations Following CAR-T Cells Transduction

Transduced CAR-T cells were incubated for 2 hours with different FasL concentrations (0, 1, 10, 50 ng/ml). Following treatment with FasL, the CAR-T cells were incubated for additional 4 days, in the presence of IL-2, for further recovery, before being analyzed.

Staining panels included T cell subtypes (naïve/CM/EM/eff cells), and additional panel of T_{H}1, T_{H}17, and T_{C}1 pro-inflammatory subtypes secreting IFNγ and IL17 that contribute to exacerbation of the pro-inflammatory reaction (during CRS and GvHD).

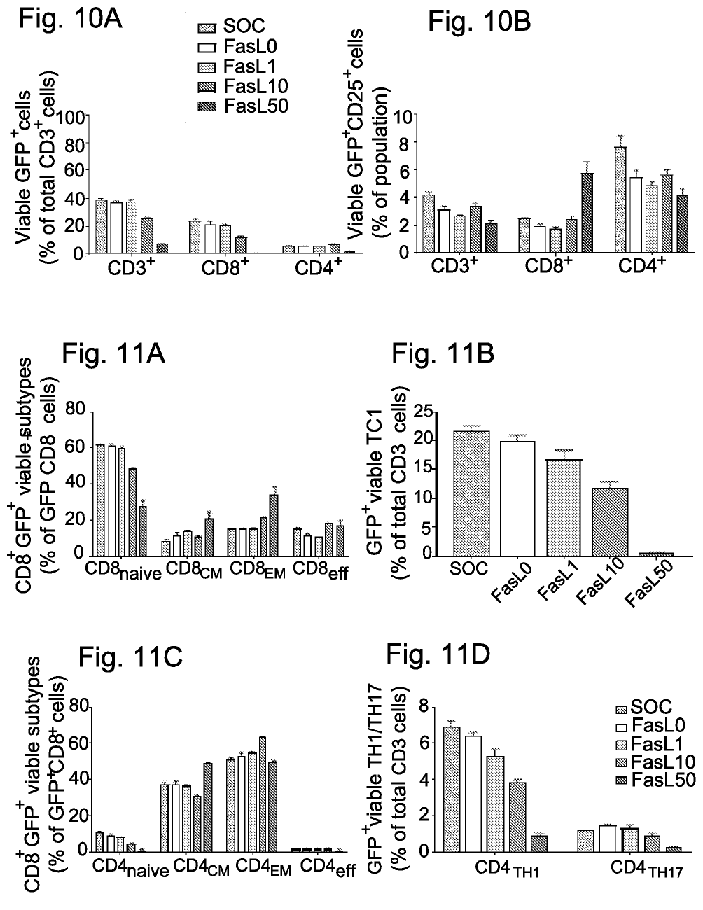

Similar to the results obtained in Experiment II, treatment of CAR-T cells post transduction with 10 and 50 ng/ml of FasL led to a reduced number of transduced cells (GFP⁺) in a dose dependent manner (FIG. 10A). During the recovery period, the cells were incubated with IL-2 with no activators, therefore the general activation state of the cells was low (FIG. 10B). The FasL treatment reduced further the activation state (as manifested by expression of CD25) of the transduced CD3⁺ cells, in comparison to the standard CAR-T (FIG. 10B), an effect that was mostly apparent in the CD4⁺ subpopulation. The remaining GFP⁺ CD8⁺ cells following exposure to 50 ng/ml FasL were highly active, as measured by the proportion of GFP⁺ cells expressing CD25 (FIG. 10B). The effect of the Fas treatment on the different T cells subtypes (naïve, central memory (CM) effector memory (EM) and effectors (eff), is depicted in FIG. 11.

1.-37. (canceled)

38. A method for producing a population of cells enriched with non-activated/non-mature cells or enriched with naïve-immune cells, comprising:
a. obtaining a biological sample comprising a heterogeneous population of mammalian cells wherein said mammalian cells are selected from the group consisting of immune cells and multipotential stromal/mesenchymal stem cells; and
b. contacting the obtained heterogeneous population of said mammalian cells with an apoptosis inducing ligand in a container,
wherein said contacting induces apoptosis of active/mature cells while non active/mature cells or naïve immune cells remain resistant to the apoptotic signal, thereby isolating a population of cells enriched for non-active/non-mature cells or naïve immune cells.

39. The method of claim 38 wherein said naïve immune cells are naïve-T cells or naïve-B cells.

40. The method of claim 38 wherein the apoptosis inducing ligand is immobilized on an inner surface of the container or on beads or films comprised in the container.

41. The method of claim 38 wherein the apoptosis inducing ligand is selected from the group consisting of TNF-α, FasL, TRAIL and TWEAK.

42. The method of claim 38 wherein said contacting step with an apoptosis inducing ligand is performed for between about 1 hour to about 48 hours, or for about 2 hours.

43. The method of claim 38 wherein said apoptosis inducing ligand is FasL and wherein said FasL is administered in a concentration of between about 1 to about 800 ng/ml, or at a concentration of about 10 ng/ml or 100 ng/ml.

44. The method of claim 38 wherein said mature cells are mature T cells selected from the group consisting of T_{H}1/T_{C}1, T_{H}17, T_{SCM}, T_{CM}, T_{EM}, and T_{eff} cell populations.

45. A population of cells enriched for naïve-T cells prepared by the method of claim 38.

46. The population of cells enriched for naïve-T cells of claim 45 wherein said cells are characterized as CCR7⁺CD45RA⁺CD95-LFA1^{low}.

47. The method of claim 38 wherein said mature cells are mature B cells selected from the group consisting of memory and plasmablast B cell populations.

48. A population of cells enriched for naïve-B cells prepared by the method of claim 38.

49. The population of cells enriched for naïve-B cells of claim 48 wherein said cells are characterized as CD27⁺CD38⁺.

50. A method of treating autoimmune diseases in a patient comprising administering to said patient a population of cells enriched for naïve-B cells or a population of cells enriched for naïve-T cells prepared by the method of claim 39.

51. A method of treating autoimmune diseases comprising:
a. contacting a heterogeneous population of mammalian immune cells comprising T and B cells with an apoptosis inducing ligand, wherein said contacting reduces the activation level of said T and B cells; and
b. administering said population of cells obtained in step (a) into a patient in need thereof.

52. A method of treating cancer in a patient comprising administering the population of cells enriched for naïve-T cells of claim 45 wherein said cells preserve their anti-cancer activity.

53. A method for producing chimeric antigen receptor (CAR)-T cells, comprising:
a. isolating mononuclear cells from a biological sample;
b. activating the cells by contacting said cells with at least one T cell activating agent; and
c. Transducing said cells with a CAR construct;
wherein said method further comprises contacting said cells with an apoptosis inducing ligand before the activating step (b) and/or after the transducing step (c), thereby obtaining CAR-T cells.

54. The method of claim 53 wherein said T cell activating agents are anti-CD3 and anti CD28 antibodies.

55. The method of claim 53 wherein the apoptosis inducing ligand is selected from the group consisting of FasL, TNF-α, TRAIL and TWEAK.

56. The method of claim 53 wherein said contacting step with an apoptosis inducing ligand is performed for between about 1 hour to about 48 hours or for about 2 hours.

57. The method of claim 53 wherein said apoptosis inducing ligand is FasL and wherein said FasL is administered in a concentration of between about 1 to about 800 ng/ml, or at a concentration of about 10 ng/ml, about 50 ng/ml or about 100 ng/ml.
